(19) United States
(12) Patent Application Publication
(10) Pub. No.: US 2009/0258404 A1
(43) Pub. Date: Oct. 15, 2009

(54) PRODUCTION OF FERMENTATION PRODUCTS IN BIOFILM REACTORS USING MICROORGANISMS IMMOBILISED ON STERILISED GRANULAR SLUDGE

(75) Inventors: Marie Just Mikkelsen, Bronshoj (DK); Birgitte Kiaer Ahring, Horsholm (DK)

Correspondence Address: ROBERTS MLOTKOWSKI SAFRAN & COLE, P.C.;Intellectual Property Department, P.O. Box 10064, MCLEAN, VA, 22102-8064, US

(73) Assignee: BIOGASOL IPR APS, Horsholm (DK)

(21) Appl. No.: 12/306,773

(22) PCT Filed: Jun. 28, 2007
(86) PCT No.: PCT/EP2007/056526

§ 371 Date: Jan. 12, 2009

(30) Foreign Application Priority Data
Jun. 30, 2006 (DK) PA 2006 00901

(51) Int. Cl. C12P 7/56; C12P 1/00; C12P 3/00; C12P 7/26; C12P 7/02; C12P 7/16; C12P 7/06; C12P 7/18; C12P 7/54; C12P 7/28

(52) U.S. Cl. 435/139; 435/41; 435/168; 435/148; 435/155; 435/160; 435/161; 435/158; 435/140; 435/150

(57) ABSTRACT

Production of fermentation products, such as ethanol and lactic acid in biofilm reactors by microorganisms immobilised on sterilised granular sludge.

### TECHNICAL FIELD

The present invention relates to microbial production of fermentation products in biofilm reactors by microorganisms immobilised on sterilised granular sludge.

### BACKGROUND OF THE INVENTION

Bioreactor systems are widely used for producing commercially valuable fermentation products such as ethanol and lactic acid, and play an important role in the biochemical industry. The systems offer high reaction rates and hence high productivity. Thus, several different types of bioreactor systems are presently being used, wherein microorganisms are grown in e.g. suspension cultures, in solid-state and immobilised-cell reactors.

In immobilised cell-reactors high microbial-cell concentrations are achieved by fixing them onto various supports. The microbial-cells can be immobilized by three different techniques; namely, adsorption, entrapment, and covalent bond formation. Entrapment and covalent bond formation require use of chemicals that add to the cost of production and perhaps restrict further propagation or increase in cell concentration inside the reactor. The third technique is of natural origin as cells “adsorb/and adhere” to the support naturally and firmly. This technique is called “adsorption” and has been used extensively to adsorb microbial cells.

In addition to being a natural process, adsorption can be performed in place, and economical adsorbents are available. Additionally, these reactors are simple in concept and construction and the immobilization process is economical. Adsorbed cells form cell layers on the support and cell mass grows inside the reactor over time. These layers of cells are called “biofilms”, and hence the reactor systems are often referred to as biofilm reactors. Biofilms can be used in various types of reactors such as continuous stirred tank reactors (CSTRs), packed bed reactors (PBRs), fluidized bed reactors (FBRs), airlift reactors (ALRs), upflow anaerobic sludge blanket (UASB) reactors, and expanded granular sludge bed (EGSB) reactors etc. In these reactors, reaction rates are usually high as compared to the other types of reactors. On the laboratory, pilot plant, and industrial scale (some), these reactors have been very successful and examples include waste water treatment and vinegar or acetic acid production. In addition to these, other processes that have employed these biofilm reactors include ethanol, butanol, lactic acid, fumaric acid, and succinic acid production.

Various types of supports have been used for adsorbing microbial cells and thereby formation of biofilms. The different types of support may be categorized into three different areas, namely inorganic support materials, encapsulation in gel-like support materials and organic support materials.

U.S. Pat. No. 5,998,185 describes a porous silicone rubber foam where microbial cells are adsorbed to the surfaces of the pores. It is described that the rubber foam may be sterilized before use and may be re-used. The structure is re-used without removing cells or is cleaned to remove cells and then re-sterilized. After loading with a culture of cells or cultures of different cells, the structure is added to a reaction medium in a bioreactor to produce a product.

U.S. Pat. No. 5,096,814 describes the immobilization of micro-organisms and animal cells in particular for anaerobic processes, such as the purification of waste water or for the biotechnological production of nutrition-essential or pharmacological substances, on porous, sintered bodies such as sintered glass in the form of Raschig rings.

U.S. Pat. No. 4,996,150 describes immobilization of microorganisms by mixing a microorganism with e.g. alginate and polyethyleneimine, and combining the resultant dispersion with an oil phase to form beads wherein the microorganisms are immobilised.

U.S. Pat. No. 4,797,358 describes the mixing of a microorganism with an alginate and a silica sol in the presence of water to obtain a liquid mixture. The mixture is subsequently contacted with a gelling agent in the form of an aqueous solution to obtain a gel containing the immobilised microorganism.

EP 1,106,679 describes the use of various kinds of organic carriers for immobilizing non-flocculent yeast, including carriers comprising chitin-chitosan, alginic acid, and carrageenan.

Schmidt et al. 1999 describes the immobilization of methanogenic bacteria on sterilized granular sludge in upflow anaerobic sludge blanket reactors. Sterile granular sludge was inoculated with either Methanosarcina mazeii S-6, Methanosaeta concilii GP-6, or both species in acetate-fed upflow anaerobic sludge blanket (UASB). No changes were observed in the kinetic parameters of the immobilized methanogens compared with suspended cultures, showing that immobilization did not affect the growth kinetics of these methanogens.

In particular continuous production of ethanol by immobilized cells has attracted much attention. Thus, Bland et al. describes (Bland R R, Chen H C, Jewell W J, Bellamy W D, Zall R R: Continuous high rate production of ethanol by Zymomonas mobilis in an attached film expanded bed fermentor. Biotechnol Lett 1982, 4:323-328) the production of ethanol in an attached film expanded bed bioreactor of Zymomonas mobilis. The cells of Z. mobilis were adsorbed onto vermiculite and the culture formed an active biofilm. Based on the total volume of the reactor, a productivity of 105 gL⁻¹h⁻¹ was obtained at a dilution rate of 3.6 h⁻¹.

Adsorbed cells of Saccharomyces cerevisiae were used in a packed bed continuous bioreactor to produce ethanol from molasses (Tyagi R D, Ghose T K: Studies on immobilized Saccharomyces cerevisiae. I. Analysis of continuous rapid ethanol fermentation in immobilized cell reactor. Biotechnol Bioeng 1982, 24:781-795). The cells were immobilized onto a support of natural origin, possibly sugarcane bagasse. It was reported that the cells were immobilized by natural mode, which is likely to be adsorption. The amount of cells that was adsorbed onto this support was 0.13 gg⁻¹ support. In this biofilm reactor, the authors reported a productivity of 28.6 gL⁻¹h⁻¹ as compared to 3.35 gL⁻¹h⁻¹ in a free cell continuous process.

Kunduru et al. studied ethanol production in continuous reactors using biofilm supports of polypropylene or plastic composite and glucose or xylose as substrate. Employing a culture of Z. mobilis and a bacterial support of polypropylene, a productivity of 536 gL⁻¹h⁻¹ was obtained at a dilution rate of 15.36 h−1. In a control free cell fermentation, a productivity of 5 gL⁻¹h⁻¹ was obtained at a dilution rate of 0.5 h⁻¹.

Recently co-fermentation of glucose-xylose mixture was studied with co-immobilization of Sacchraromyces cerevisiae with a xylose fermenting yeast Pichia stipitis in calcium-alginate beads in CSTR (continuous stirred tank reactor) and FBR (fluidized-bed bioreactor) (De Bari et al., 2004). In both reactor configurations (FBR and CSTR), ethanol production was mainly due to the glucose fermentation.

Recombinant Zymomonas mobilis CP4 (pZB5) immobilized in k-carrageenan beads in FBR was able to convert various mixtures of glucose and xylose with high ethanol yields (0.33-0.43 g/g based on available sugars) and high volumetric productivities (6.5-15 g/l/h). However, it was seen that at long-term reactor operation, the organism gradually lost the plasmid carrying the genes encoding xylose metabolism enzymes resulting in a decrease in xylose conversion to 10%.

Because of industrial potential benefits offered by fermentation at elevated temperatures attempts have also been made to exploit cell immobilization for thermophilic ethanol production. Recently ethanol production from glucose and molasses at 45° C. by thermotolerant yeast strain Kluyveromyces marxianus IMB3 immobilized in calcium alginate and kissiris was reported, and it was demonstrated that the immobilization increased the ethanol productivity for the variety of bioreactor configurations tested (Gough et al. 1997). Liu et al. (1988) have studied the conversion of xylose (6 g/l) by immobilized cells of anaerobic bacterium Clostridium thermosaccharolyticum on polystyrene chips in continuous up-flow reactor at 60° C., nevertheless, using immobilized culture was not advantageous and sub-optimal productivity was found compared to free batch culture.

Although promising, several of the above methods for immobilizing microorganisms face the common technical problem that the performance of the microorganisms, and thereby the yield of the end-product, may be sub-optimal due to the microorganisms being exposed to high substrate concentrations in the biofilm reactors. Another common problem is that the productivity of the fermenting microorganisms may also be significantly restrained due the inhibitory effect of the concentration of the resulting end-product. High end-product concentrations are of particular importance when the microorganisms are for alcohol production such as ethanol production, since e.g. distillation costs increase with decreasing concentrations of alcohol. In general, the ethanol tolerance of thermophilic Clostridia is low (typically less than 2% w/w). Although mutant strains have been obtained, which are tolerant up to 10% of ethanol, but promising continuous fermentations with these mutants have not been demonstrated. Continuous ethanol production using Clostridium thermosaccharolyticum has been reported at 3.7% w/w of ethanol in the fermentation medium. Hence, improved methods for improving tolerance to high concentrations fermentation end-products, such as ethanol, are highly needed.

It has now been surprisingly been found that it is possible to significantly increase the effectiveness of carbohydrate solution fermentation in biofilm reactors, by immobilizing microorganisms onto sterilized sludge and thereby significantly increase the yield of fermentation end-products such as alcohols and organics acids. In particular it has been found by the present inventors, that ethanol may by produced by the use of anaerobic thermophilic microorganisms which are normally not suitable for continuous ethanol production due their inherent low ethanol tolerance. As will also be apparent from the following, the technique is highly useful when using immobilized thermophilic anaerobes for bioconversion of lignocellulosic hydrolysate into ethanol.

### SUMMARY OF THE INVENTION

Accordingly, the present invention pertains to a process for the continuous production of a fermentation product by a fermentation process, wherein the fermentation product is produced by fermenting a carbohydrate solution in a biofilm reactor. The process comprises the steps of (i) providing a solid support comprised of sterilised granular sludge, (ii) admixing the solid support with an appropriate liquid medium comprising a carbohydrate solution to form a liquid growth medium, (iii) contacting said liquid growth medium with an amount of microorganisms for a time period effective for cells of the microorganisms to attach to the surface of said solid support and form a film of microorganism cells on a substantial proportion of the surface of the support, and (iv) cultivating the microorganisms under appropriate conditions to produce the fermentation product.

### DETAILED DESCRIPTION OF THE INVENTION

As mentioned above, the present invention relates to a process for the continuous production of a fermentation product by a fermentation process wherein the fermentation product is produced by fermenting a carbohydrate solution in a biofilm reactor.

Biofilm reactors are microbial cell-reactors wherein high microbial-cell concentrations are achieved by immobilising the cells onto various supports. The immobilised cells form cell layers on the support and cell mass grows inside the reactor over time, and these layers of cells are called “biofilms”. Biofilms are, as mentioned above used in various types of immobilised cell-reactors such as continuous stirred tank reactors, packed bed reactors, fluidized bed reactors, airlift reactors, upflow anaerobic sludge blanket reactors, and expanded granular sludge bed reactors, etc. These types of reactors have traditionally been used for vinegar or acetic acid production, but are increasingly being used for the production of other fermentation products.

Numerous fermentation products are valuable commodities which are utilised in various technological areas, including the food industry and the chemical industry.

Lactic acid is extensively used in the cosmetics industry as an anti-aging chemical, and the food industry use lactic acid in a variety of food stuffs to act as an acidity regulator. Recently, lactic acid has also attracted much attention for its potential use in biodegradable polyesters.

Hydrogen is widely used in the petroleum and chemical industries, i.a. for the processing of fossil fuels, for hydroalkylation, hydrodesulfurization and hydrocracking, and it is used for the hydrogenation of fats and oils (found in items such as margarine), and in the production of methanol. Additionally, hydrogen can be used as an energy source, and can be burned in e.g. combustion engines.

Acetic acid is a valuable product which is widely used in industry, mainly for the production of vinyl acetate monomer, ester production, vinegar, and for use as a solvent. The global demand of acetic acid is around 6.5 million tonnes per year.

Due to the increasing global energy requirements and air pollution caused by green house gases, ethanol has lately received particular attention as a potential replacement for or supplement to petroleum-derived liquid hydrocarbon products, and particularly ethanol derived from plant materials (bioethanol).

Other valuable fermentation products includes butanol, fumaric acid, and succinic acid.

As mentioned previously, the yield of resulting fermentation end-products are often to low to make the fermentation processes economically feasible. Therefore there is a high need for improvement of fermentation processes performed in biofilm reactors.

It has been found by the present inventors, that it is possible to significantly increase the effectiveness of continuous fermentation of carbohydrate solution in biofilm reactors, by immobilizing microorganisms onto sterilized granular sludge and thereby increase the yield of fermentation end-products such as alcohols and organics acids. Thus, as will be apparent from the following examples, continuous fermentations were performed at high concentrations of sugars with high ethanol productivity and yield as shown in example 1 and 2. By applying the process of the present invention, problems with toxicity of lignocellulosic hydrolysates have been overcome as shown in examples 3, 4 and 5 on three different kinds of un-detoxified lignocellulosic hydrolysates. Finally, although the thermophilic Clostridia used in the experiments inherently have a limited ethanol tolerance (typically less than 2% w/w) of, fermentations could be performed using liquid media with high ethanol concentrations even up to 8.3% w/w.

Accordingly, the present invention provides a process for continuous production of a fermentation product by a fermentation process, wherein the fermentation product is produced by fermenting a carbohydrate solution in a biofilm reactor. The process comprises the steps of (i) providing a solid support comprised of sterilised granular sludge, (ii) admixing solid support with an appropriate liquid medium comprising a carbohydrate solution to form a liquid growth medium, (iii) contacting the liquid growth medium with an amount of microorganisms for a time period effective for cells of the microorganisms to attach to the surface of said solid support and form a film of microorganism cells on a substantial proportion of the surface of the support, and (iv) cultivating the microorganisms under appropriate conditions to produce the fermentation product.

The solid support is composed of sterilized granular sludge which serves for immobilising the microbial cells in the biofilm reactor. Granular sludge may advantageously be obtained from already operating bioreactors, such as fluidized bed reactors (FBR), gaslift reactors, upflow anaerobic sludge blanket (UASB) reactors, upflow staged sludge bed (USSB) reactors, expanded granular sludge bed (EGSB) reactors, internal circulation reactors, upflow anaerobic filter processes (UAFP), and anaerobic fluidized-bed reactors (AFBR). The biomass in such reactor types is retained as aggregates called granules, formed by self-immobilisation or conglomeration of the bacteria. In UASB reactors, the granular sludge is located in the bottom of the reactor (the granular sludge bed), and it is also here organic compounds are biologically degraded to methane and carbon dioxide as the microbial community will be complex under these non-sterile conditions.

Typically, the operating biofilm reactors from where the granular sludge is obtained, are or have been processing waste water with a high content of organic material such as municipal waste water, paper mill waste water, waste water from production of food products such as potatoes, sugar or starch, waste water from the production of organic compounds such as citric acid or lactic acid, production of oils and fats, or waste water from malting, brewing or distilling processes.

In accordance with the invention the diameter of the sludge granules may vary from about 0.1 to about 5 mm, including the range of about 2 to 4 mm, depending upon the origin of the granular sludge and the operational conditions of the bioreactors from where the granular sludge was obtained. Thus, granules which have been cultivated on acidified substrates, such as acetate, are generally smaller than granules grown on acidogenic substrates, e.g., glucose. The size of the sludge granules may be determined visually e.g. by using a microscope equipped with appropriate measuring devices. The granules may vary widely in shape, depending on the conditions in the bioreactor; but they usually have a spherical form.

Andras et al. 1989 have developed a test to characterize the settleability of granular sludge. The test is based on the division of sludge into fractions depending on their resistance to wash-out of a test cylinder with increasing linear flow. A test like this accounts for both the buoyant density, the shape, and the volume of the granules. Granules with high buoyant densities and volume will wash out at higher linear flow rates compared to small granules with low buoyant densities. The linear liquid flow rate at which a granule with a given volume and buoyant density will be washed out of the reactor can be estimated by Stoke's law. Granules with different volumes and densities can be present in a reactor at a given linear flow rate; both small granules with high densities and larger granules with low and high densities will be present. Reported settling velocities for granular sludge are in the range of 18 to 100 m/h, but typical values are between 18 and 50 m/h. Granular sludge can therefore be divided into three fractions based on the reported settling velocities: a poor settling fraction, a satisfactorily settling fraction, and a good settling fraction, with settling velocities of up to 20 m/h, from 20 to 50 m/h, and over 50 m/h, respectively. A satisfactory granular sludge contains sludge with its main part in the two last fractions. Hence, in accordance with the invention the sterilised granular sludge is preferably comprised of spherical granules having a settling velocity in the range of about 18 to 100 m/h., including up to about 20 m/h, such as from about 20 to 50 m/h, including at least about 50 m/h, when using the settleability test for granular sludge described by Andras et al. 1989.

The volatile suspended solids value of the sterilised granular sludge is preferably in the range of 5-15%, such as in the range of 7-12% including the range of 8-11%. Volatile suspended solids are the solids that are removed by firing a sample in a 550° C. muffle furnace.

The process according to invention is typically performed in a biofilm reactor such as a fluidized bed reactor (FBR), a gaslift reactor, an upflow anaerobic sludge blanket reactor (UASBR), an upflow staged sludge bed (USSB) reactor, an expanded granular sludge bed (EGSB) reactor, an internal circulation reactor, and an upflow anaerobic filter process (UAFP).

The granular sludge is sterilised before mixing it with the liquid growth medium, and the sterilisation may be performed by any suitable methods, including autoclavation, and the use of ionizing radiation. In certain embodiments, it may be necessary to sterilise the granular sludge more than once, e.g. when using autoclavation, in order to kill the microorganisms completely. The sterilisation of the granular sludge may for certain embodiments be performed directly in the biofilm reactor, before admixing it with the liquid growth medium.

The carbohydrate solution serves as the substrate for the immobilised microorganisms. In the present context the term “carbohydrate solution” is intended to include solutions comprising chemical compounds having the general chemical formula Cₙ(H₂O)ₙ. Thus, the term “carbohydrate” includes monosaccharides, oligosaccharides and polysaccharides as well as substances derived from monosaccharides by reduction of the carbonyl group (alditols, including sugar alcohols such as glycerol, mannitol, sorbitol, xylitol and lactitol, and mixtures thereof), by oxidation of one or more terminal groups to carboxylic acids, or by replacement of one or more hydroxy group(s) by a hydrogen atom, an amino group, a thiol group or similar heteroatomic groups. It also includes derivatives of these compounds.

The generic term “monosaccharide” (as opposed to oligosaccharide or polysaccharide) denotes a single unit, without glycosidic connection to other such units. It includes aldoses, dialdoses, aldoketoses, ketoses and diketoses, as well as deoxy sugars and amino sugars, and their derivatives, provided that the parent compound has a (potential) carbonyl group. The term “sugar” is frequently applied to monosaccharides and lower oligosaccharides. Typical examples are glucose, fructose, xylose, arabinose, galactose and mannose.

“Oligosaccharides” are compounds in which monosaccharide units are joined by glycosidic linkages. According to the number of units, they are called disaccharides, trisaccharides, tetrasaccharides, pentasaccharides etc. The borderline with polysaccharides cannot be drawn strictly; however the term “oligosaccharide” is commonly used to refer to a defined structure as opposed to a polymer of unspecified length or a homologous mixture. Examples are sucrose and lactose.

“Polysaccharides” is the name given to a macromolecule consisting of a large number of monosaccharide residues joined to each other by glycosidic linkages.

In accordance with the invention the polysaccharides may be selected from starch, lignocellulose, cellulose, hemicellulose, chitin, pectin, glycogen, xylan, glucuronoxylan, arabinoxylan, arabinogalactan, glucomannan, xyloglucan, and galactomannan.

As will be apparent from the following examples, by applying the method according to the invention using certain thermophilic microorganisms, fermentation products may be produced on very high dry-matter concentrations of lignocellulosic hydrolysates. In the present context the term “lignocellulosic hydrolysate” is intended to designate a lignocellulosic biomass which has been subjected to a pre-treatment step whereby lignocellulosic material has been at least partially separated into cellulose, hemicellulose and lignin thereby having increased the surface area of the material. Useful lignocellulosic material may, in accordance with the invention, be derived from plant material, such as straw, hay, garden refuse, house-hold waste, wood, fruit hulls, seed hulls, corn hulls, oat hulls, soy hulls, corn fibres, stovers, milkweed pods, leaves, seeds, fruit, grass, wood, paper, algae, cotton, hemp, flax, jute, ramie, kapok, bagasse, mash, distillers grains, oil palm, corn, sugar cane and sugar beet.

The pre-treatment method most often used is acid hydrolysis, where the lignocellulosic material is subjected to an acid such as sulphuric acid whereby the sugar polymers cellulose and hemicellulose are partly or completely hydrolysed to their constituent sugar monomers. Another type of lignocellulose hydrolysis is steam explosion, a process comprising heating of the lignocellulosic material by steam injection to a temperature of 190-230° C. A third method is wet oxidation wherein the material is treated with oxygen at 150-185° C. The pre-treatments can be followed by enzymatic hydrolysis to complete the release of sugar monomers. This pre-treatment step results in the hydrolysis of cellulose into glucose while hemicellulose is transformed into the pentoses xylose and arabinose and the hexoses glucose, galactose and mannose. The pre-treatment step may in certain embodiments be supplemented with treatment resulting in further hydrolysis of the cellulose and hemicellulose. The purpose of such an additional hydrolysis treatment is to hydrolyse oligosaccharide and possibly polysaccharide species produced during the acid hydrolysis, wet oxidation, or steam explosion of cellulose and/or hemicellulose origin to form fermentable sugars (e.g. glucose, xylose and other monosaccharides). Such further treatments may be either chemical or enzymatic. Chemical hydrolysis is typically achieved by treatment with an acid, such as treatment with aqueous sulphuric acid, at a temperature in the range of about 100-150° C. Enzymatic hydrolysis is typically performed by treatment with one or more appropriate carbohydrase enzymes such as cellulases, glucosidases and hemicellulases including xylanases.

The above pre-treatment processes all share the same general problem, namely the generation of degradation products such as furfural, phenols and carboxylic acids, which can potentially inhibit the fermenting organism. The inhibitory effect of the hydrolysates can be reduced by applying a detoxification process prior to fermentation, but the inclusion of this extra process step increases significantly the total cost of the fermentation product and should preferably be avoided. However, by applying the process according to the invention, the inhibitory effect of the degradation products may be minimised significantly, as the immobilised microorganisms will not be subjected to the same concentration of degradation products as microorganisms in suspended cultures.

It has also been found that the process according to the invention in useful embodiments may be performed using a liquid growth medium comprising a hydrolysed lignocellulosic biomass material having a dry-matter content of at least 10% wt/wt, such as at least 15% wt/wt, including at least 20% wt/wt, such as at least 25% wt/wt and even as high as at least 35%. This has the great advantage that it may not be necessary to dilute the hydrolysate before the fermentation process, and thereby it is possible to obtain higher concentrations of fermentation products such as ethanol, and thereby the costs for subsequently recovering the fermentation products may be decreased. For example the distillation costs for ethanol will increase with decreasing concentrations of alcohol.

Ethanol production from plant materials (lignocellulosic biomass) has attracted widespread attention as an unlimited low cost renewable source of energy for transportation fuels. Because the raw material cost accounts for more than 30% of the production costs, economically, it is essential that all major sugars present in lignocellulosic biomass are fermented into ethanol. The major fermentable sugars derived from hydrolysis of various lignocellulosic materials are glucose and xylose. Microorganisms currently used for industrial ethanol production from starch materials, Saccharomyces cerevisiae and Zymomonas mobilis, are unable naturally to metabolize xylose and other pentose sugars. Considerable effort has been made in the last 20 years in the development of recombinant hexose/pentose-fermenting microorganisms for fuel ethanol production from lignocellulose sugars, however, a common problem with genetically engineered ethanologens is co-fermentation of glucose with other sugars, known as “glucose repression” i.e. sequential sugar utilization, xylose conversion starts only after glucose depletion, resulting in “xylose sparing” i.e. incompletely xylose fermentation. Co-fermentation of glucose and xylose is therefore a crucial step in reducing ethanol production cost from lignocellulosic raw materials. Thermophilic anaerobic bacteria have the unique trait of being able to ferment the whole diversity of monomeric sugars present in lignocellulosic hydrolysates. In addition, the industrial use of thermophilic microorganisms for fuel ethanol production offers many potential advantages including high bioconversion rates, low risk of contamination, cost savings via mixing, cooling and facilitated product recovery. These microorganisms are, however, sensitive to high ethanol concentrations and produce low ethanol yields at high substrate concentrations.

However, as will be seen from the following examples, the present method allows for high yield production of ethanol by microorganisms which are normally very sensitive to high ethanol concentrations. Thus, it was found that Thermoanaerobacter BG1 which under batch conditions has been found to have an absolute limit for growth at an ethanol concentration of 4% v/v, could ferment xylose and continue producing at ethanol concentrations of 8.3% v/v.

As mentioned previously, one of the steps in the process according to the invention is the mixing of the solid support (the sterilised granular sludge) with the carbohydrate solution to form a liquid growth medium for the microorganisms. The liquid growth medium may additionally comprise other compounds such as minerals, vitamins and pH-adjusting agents, depending on the specific requirements of the selected microorganisms.

Subsequent to the step of forming the liquid growth medium comprising the solid support in the form of sterilised granular sludge, the liquid growth medium is contacted with an amount of microorganisms for a time period effective for cells of the microorganisms to attach to the surface of the solid support and form a film or a thin coating of microorganisms on a substantial proportion of the surface of the support. Preferably, the solid support is held in contact with the microorganisms for a time period effective for the microorganisms to grow over the surface of the support and form a coherent film over a substantial portion of the support surface.

A “pure culture” biofilm reactor may be formed by immobilizing a culture of single microorganism onto the support. Alternatively, a “mixed culture” biofilm reactor may be prepared by first immobilizing cells of a biofilm-forming microorganism on the surface of the support, and then immobilizing a non- or low-film forming microorganism onto the cells of the biofilm-forming microorganism on the support.

Suitable microorganisms for producing fermentation end-products and which may attach to and form a film or coating layer on the surface of the support include, for example, a microorganism belonging to a genus selected from Saccharomyces, Thermoanaerobacter, Clostridium, Moorella, Lactobacillus, Aspergillus, Pichia, Zymomonas, Zymobacter, Pseudomonas, Escherichia, Acetobacterium, Propionibacterium og Acetogenium. In useful embodiments the microorganism belonging to genus of Thermoanaerobacter may be selected from Thermoanaerobacter acetoethylicus, Thermoanaerobacter brockii, Thermoanaerobacter brockii subsp. brockii, Thermoanaerobacter brockii subsp. finnii, Thermoanaerobacter brockii subsp. lactiethylicus, Thermoanaerobacter ethanolicus, Thermoanaerobacter finnii, Thermoanaerobacter italicus, Thermoanaerobacter kivui, Thermoanaerobacter lacticus, Thermoanaerobacter mathranii, Thermoanaerobacter pacificus, Thermoanaerobacter siderophilus, Thermoanaerobacter subterraneus, Thermoanaerobacter sulfurophilus, Thermoanaerobacter tengcongensis, Thermoanaerobacter thermocopriae, Thermoanaerobacter thermohydrosulfuricus, Thermoanaerobacter wiegelii, and Thermoanaerobacter yonseiensis.

In specific embodiments the Thermoanaerobacter mathranii strain is HY10 (DSMZ Accession number 14578) or BG1 (DSMZ accession number 18280) which have been found to particularly suited for the production of acetate, lactate and ethanol in upflow immobilized reactors by fermentation of xylose. The Thermoanaerobacter mathranii mutant strain BG1L1 (DSMZ accession number 18283) derived from BG1 was found to be very useful for the continuous production of ethanol by fermentation of xylose and co-fermentation of xylose and glucose sugars in an upflow immobilized reactor. It is contemplated that other mutants of Thermoanaerobacter mathranii BG1 may advantageously be used in the present invention, including the strains BG1PF1 (DSMZ Accession number 18282) and BG1H1 (DSMZ Accession number 18281).

In accordance with the invention, the microorganisms are cultivated under appropriate conditions to produce the fermentation product. Hence, conditions such as pH, temperature, and dilution rate may be adjusted so as to optimise the production rate of the fermentation product. In useful embodiments, the fermentation process is performed under strict anaerobic conditions.

As will be seen from the following examples, the process according to the invention is highly useful for performing fermentation processes using thermophilic microorganisms. Hence, the process may be operated at a temperature in the range of about 40-95° C., such as the range of about 50-90° C., including the range of about 60-85° C., such as the range of about 65-75° C.

The Hydraulic retention time (HRT) is a measure of the average length of time that a soluble compound remains in the biofilm reactor. When performing the process according to the invention in a an upflow immobilized reactor, it is preferably operated at a hydraulic retention time in the range of about 2 to 75 hours, including the range of 7 to 12 hours, including 8 to 10 hours, including 20-30 hours.

In accordance with the invention, the method is useful for the production of a wide range of fermentation products including acids, alcohols, ketones and hydrogen. Thus fermentation products such as ethanol, butanol, propanol, methanol, propanediol, butanediol, lactic acid, proprionate, acetate, succinate, butyrate, formate and acetone may be produced in accordance with the invention.

The process according to the invention may optionally comprise a recovery step for retrieval of the fermentation product. When alcohol (e.g. ethanol) is the fermentation product, it may be advantageous to use steam or gas stripping for the recovery of the alcohol simply by blasting steam or gas (such as CO₂) through the fermentation broth, collecting the vapour, and either condensing it or feeding it to a distillation system. Alternatives are membrane processes or solvent extraction

The invention will now be further described in the following non-limiting examples and figures.

### SHORT DESCRIPTION OF THE FIGURES

FIG. 1: Schematic outline of the lab-scale upflow immobilized reactors used in the experiments.

FIG. 2: Start-up and running of an upflow immobilized reactor with Thermoanaerobacter HY10 at 10 g/l of xylose and decreasing HRT.

FIG. 3: Reactor performance of an upflow immobilized reactor with Thermoanaerobacter HY10 at extreme loadings, shock and recovery.

FIG. 4: Performance of two upflow immobilized reactors with Thermoanaerobacter HY10 at different hydraulic retention times (HRT) and organic loading rates, calculated as average from the two reactors.

FIG. 5: Time course of continuous ethanol fermentation from xylose (10 g/l) at various HRT using immobilized cells of Thermoanaerobacter BG1L1 at 70° C. and no pH control

FIG. 6: Summary of ethanol production from xylose (10 g/l) by immobilized cells of Thermoanaerobacter BG1L1 in an upflow immobilized reactor at 70° C. and no pH control

FIG. 7: Summary of ethanol production from glucose-xylose mixtures by immobilized cells of Thermoanaerobacter BG1L1 in an upflow immobilized reactor at 70° C. and pH=7.

FIG. 8: Glucose and xylose concentrations in various steam exploded and enzyme treated wheat straw hydrolysate suspensions.

FIG. 9: Product yields obtained with Thermoanaerobacter BG1L1 in an upflow immobilized reactor at 70° C. from steam exploded and enzyme treated wheat straw hydrolysate suspensions.

FIG. 10: Influent sugar concentrations (A) and sugar conversions (B) for various acid hydrolyzed corn stover hydrolysate suspensions in an upflow immobilized reactor with Thermoanaerobacter BG1L1 at 70° C., pH 7.

FIG. 11: Effluent product concentrations (acetate and ethanol) and influent acetate concentration for various acid hydrolyzed corn stover hydrolysate suspensions from continuous fluidized bed reactor with immobilized Thermoanaerobacter BG1L1 at 70° C., pH 7.

FIG. 12: Ethanol yield and carbon recovery for various acid hydrolyzed corn stover hydrolysate suspensions in an upflow immobilized reactor with Thermoanaerobacter BG1L1 at 70° C., pH 7.

FIG. 13: Influent sugar concentrations and sugar conversions for various wet oxidized and enzyme treated wheat straw hydrolysate suspensions for an upflow immobilized reactor with Thermoanaerobacter BG1L1 at 70° C., pH 7.

FIG. 14: One-step fermentation of wet oxidized and enzyme treated wheat straw by Thermoanaerobacter BG1L1

FIG. 15: Ethanol yields obtained at various wet oxidized and enzyme treated wheat straw hydrolysate suspensions from an upflow immobilized reactor with Thermoanaerobacter BG1L1 at 70° C.

FIG. 16: Effect of exogenously added ethanol on xylose conversion and product formation by immobilized cells of Thermoanaerobacter BG1L1 in a FBR with no pH control at 70° C. The xylose concentration in the feed was 10 g/l.

### EXAMPLES

### Materials and Methods

The following materials and methods were applied in the below Examples:

### Strains and Growth Conditions

Strain BG1 (DSMZ accession number 18280; DSMZ—Deutsche Sammlung von Mikroorganismen und Zellkulturen GmbH, Mascheroder Weg 1b, 38124 Braunschweig, Germany) is a Thermoanaerobacter isolated anaerobically from an Icelandic hot-spring at 70° C. BG1L1 (DSMZ accession number 18283; DSMZ—Deutsche Sammlung von Mikroorganismen und Zellkulturen GmbH, Mascheroder Weg 1b, 38124 Braunschweig, Germany) is a lactate dehydrogenase deficient mutant of BG1. HY10 (DSMZ accession number 14578; DSMZ—Deutsche Sammlung von Mikroorganismen und Zellkulturen GmbH, Mascheroder Weg 1b, 38124 Braunschweig, Germany) is a mutant strain of a Thermoanaerobacter strain isolated anaerobically from an Icelandic hot-spring at 70° C.

All strains were cultured at 70° C. anaerobically in minimal medium (BA) with 2 g/l yeast extract as in (Larsen et al., 1997) unless otherwise stated.

### Continuous Reactors

Fermentation medium used for continuous cultivation was prepared and supplemented with the same minerals, trace metals, and yeast extract as described above unless otherwise stated. The initial pH of the medium was adjusted to 7.4-7.7 and it was autoclaved at 120° C. for 30 min. To ensure anaerobic conditions, medium was flushed for 45 minutes with a mixture of N2/CO₂ (4:1), and finally Na₂S was injected into the bottle to give a final concentration of 0.25 g/l.

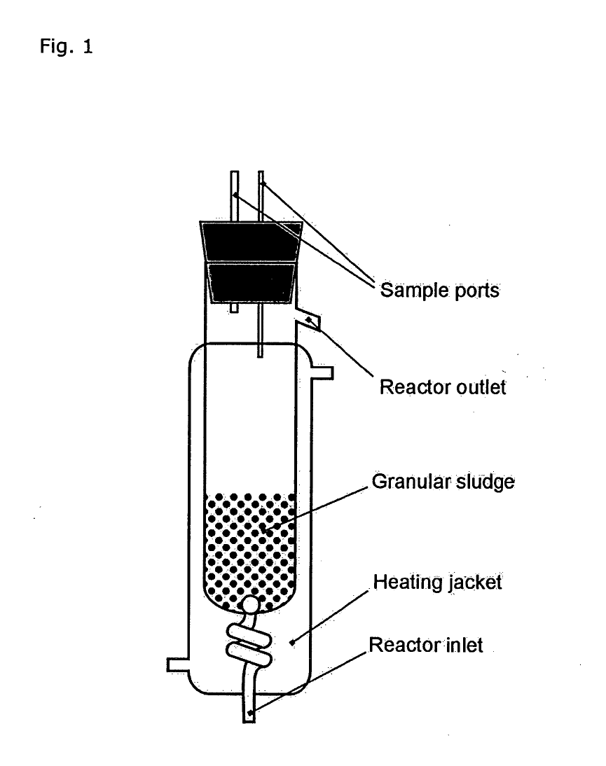

The reactor was a water-jacketed glass column with 4.2 cm inner diameter and 20 cm height (FIG. 1). The working volume of the reactor was 200 ml. The influent entered from the bottom of the reactor and the feeding was controlled by a peristaltic pump (Model 5035-10 rpm, Watson Marlow, Falmouth, UK). Recirculation flow was achieved by using an identical peristaltic pump (Model 503-50 rpm, Watson Marlow, Falmouth, UK), with a degree of recirculation to ensure up-flow velocities in the reactor of 1 m/h. The pH was maintained at 7.0 by addition of NaOH (1-2M), unless otherwise stated. The reactor was loaded with 75-ml of sterilized granular sludge originating either from a paper mill factory in the Netherlands, Eerbeek BV., or from the UASB reactor at Faxe waste water treatment plant (Denmark), and finally the entire reactor system, including the tubing and recirculation reservoir, was autoclaved at 120° C. for 30 min. Before use, the reactor system was gassed for 15 minutes with N₂/CO₂ (4:1) to ensure anaerobic conditions and filled with BA medium with an initial xylose concentration of 10 g/l. The reactor was started up in batch mode by inoculation with 80 ml of cell suspension with an optical density (OD₅₇₈) of 0.9-1. The batch mode of operation was maintained for 24 hours to allow cells to attach and to immobilize on the carrier matrix. After the batch run, the system was switched to continuous mode applying a HRT of 8 hours and an up-flow velocity of 1 m/h.

Liquid samples were taken from sampling ports located on the top of the reactor, close to the reactor outlet. The experiments were performed at 70° C. by external heating and recirculation of hot water in the glass jacket.

During the experiments, whenever steady state was achieved, HRT or sugar concentrations were changed. The criteria for steady-state conditions were, that all parameters must be held constant for at least five residence times. The reactor performance at different steady state was monitored by measuring the sugar and end-fermentation product concentrations. During the experiment, sterile syringes and needles were used to take the samples from the influent and effluent, and the samples were stored at −20° C. until analyzing. Effluent gas samples were taken to determine the carbon dioxide and hydrogen content.

### Test for Contamination

A 1 ml sample was taken from the reactor and chromosomal DNA was purified using the DNA purification kit from A&A Biotech (Poland). PCR reactions were setup using the Pfu polymerase (MBI Fermentas, Germany) and the primers B-all 27F and B-all 1492R, which anneal to bacterial rDNA at the 5′-end and 3′-end respectively. The fragments were purified using the Qiaex II kit from Qiagen, treated with PNK (MBI Fermentas), cloned into pBluescript SK+ (Stratagene) treated with CIAP (MBI Fermentas), and transformed into Escherichia coli Top10 (Invitrogen). 50 clones were picked and the inserts were amplified using B-all 27F and B-all 1492R primers. The resulting fragments were digested with AluI and MboI restriction enzymes (MBI Fermentas) and were run on a 3% agarose gel. Only one digestion pattern was found. Two fragments were sent for sequencing (MWG Biotech, Germany) and were identified as strain BG1. PCR reactions were also run using primers ldhcw1 and ldhccw2 annealing to regions upstream and downstream of the lactate dehydrogenase respectively. Otherwise, the same reaction conditions as for the B-all primers, were used. The obtained fragments were cloned (as above), 26 were analysed by restriction fragment length polymorphism. Again, this resulted in only one pattern. Two fragments were sequenced.

### Enzymes and Reagents

If not stated otherwise enzymes were supplied by MBI Fermentas (Germany) and used according to the suppliers' recommendations. PCR reactions were performed with a 1 unit:1 unit mixture of Taq polymerase and Pfu polymerase. Chemicals were of molecular grade and were purchased from Sigma-Aldrich Sweden AB.

### Analytical Techniques

The culture supernatants were analyzed for cellobiose, glucose, xylose, acetate, lactate and ethanol using an organic acid analysis column (Aminex HPX-87H column (Bio-Rad Laboratories, CA USA)) on HPLC at 65° C. with 4 mM H₂SO₄ as eluent. The ethanol and acetate measurements were validated using gas chromatography with flame ionization detection. Mixed sugars were measured on HPLC using a Phenomenex, RCM Monosaccharide (00H-0130-K0) column at 80° C. with water as eluent. Hydrogen was measured using a GC82 Gas chromatograph (MikroLab Aarhus, Denmark).

### Yield and Carbon Recovery

Theoretical maximum yields and carbon recoveries were calculated based on the following reactions (ATP and NAD(P)+ conversions are not included):

1 M glucose→2 M lactate,

1 M glucose→2 M acetate+2 M CO₂,

1 M Glucose→2 M Ethanol+2 M CO₂

3 M Xylose→5 M Lactate,

3 M Xylose→5 M Acetate+5 M CO₂,

3 M Xylose→5 M Ethanol+5 M CO₂

The theoretical maximum yields of ethanol from glucose and xylose are therefore 2 and 1.67 moles per mole respectively.

Carbon recoveries were calculated as:

$$\frac{3 \times ( mM lactate + mM acetate + mM ethanol produced )}{n \times ( mM substrate consumed )} \times 100 \%$$

where n is 5 for xylose and 6 for glucose.

### Example 1

### Immobilised Thermoanaerobacter HY10: Continuous Production of Acetate, Lactate and Ethanol in UIR Reactors by Fermentation of Xylose

Two Upflow immobilized reactors (UIR) were operated with the thermophilic anaerobic bacterium Thermoanaerobacter HY10 at gradually decreasing hydraulic retention times. The carrier material in the reactors originated from a mesophilic full-scale UASB reactor digesting wastewater from a paper mill factory in the Netherlands, Eerbeek BV. The granular sludge was sterilized conducting 3 cycles of autoclaving at 130° C. for 20 minutes followed by overnight incubation at 37° C. After the last cycle 75 mL of granular sludge was transferred to each reactor. The granular sludge transferred to the reactors had a total suspended solid (TSS) content of 9.8% (w/w) and a volatile suspended solids (VSS) content of 6.4% (w/w). Finally the entire reactor systems, including tubing and recirculation reservoirs, were autoclaved at 120° C. for 30 minutes.

After cooling, the reactors were filled with anaerobic cultivation medium with an initial xylose concentration of 10 g/L. Subsequently, the reactors were started up in batch mode by inoculation with 5% (v/v) Thermoanaerobacter HY10 batch culture having a cell density corresponding to 0.4 g-TS/L. The inoculum culture of T. HY10 was prepared in 50 mL bottles containing cultivation medium with 5 g-xylose/L, incubated at 70° C. The culture used for inoculation originated from a frozen stock culture transferred and cultivated three times using the mineral media amended with 5 g-xylose/L. After 24 hr of batch-run, the recirculation and feed pumps were turned on, applying a vertical flow rate of 1 m/hr and a HRT of 16 hr. The influent xylose concentration was 10 g/L corresponding to an organic loading rate (OLR) of 16 g-COD/(L*d) (COD=chemical oxygen demand, L=litre, d=day). During the experiment, the HRT was gradually lowered to 1 hr corresponding to 245 g-COD/(L*d) under constant up-flow velocity of 1 m/hr. The reactor performances at different steady states were monitored by measuring the pH level and xylose, ethanol, lactate, and acetate concentrations on a daily basis. Effluent gas samples were taken to determine the hydrogen and carbon dioxide content, throughout the experiments.

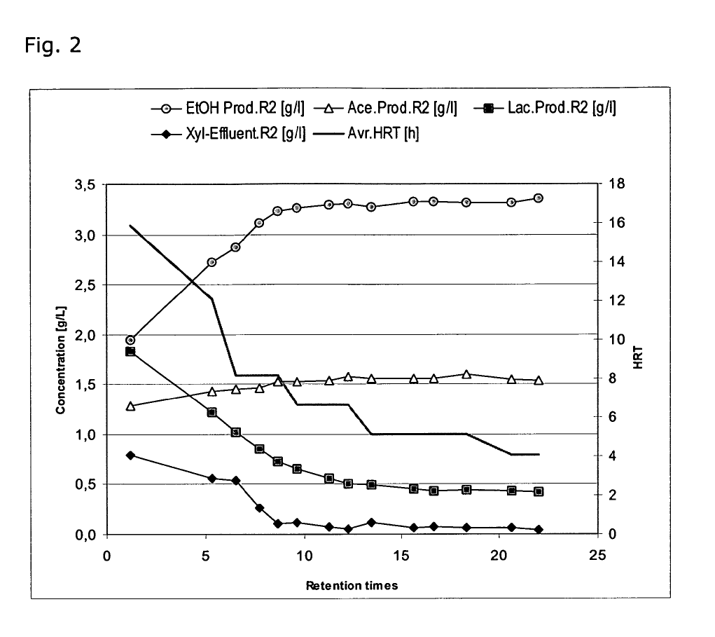

The two UIR reactors were started up at a HRT of 16 hr corresponding to an OLR of 16 g-COD/(L*d) (FIGS. 2, 3 and 4). Whenever steady state was observed as similar consecutive measurements of product and xylose concentrations for more than 2 hydraulic retention times, the HRT was decreased stepwise. The two reactors showed similar behaviour during the startup phase. In FIG. 2 the HRT and the concentrations of ethanol, acetate, lactate and residual xylose are shown for the startup of Reactor 2 (R2). Preliminary experiments carried out in a chemostat with Thermoanaerobacter HY10 fermenting xylose in a similar cultivation media showed that the minimum achievable HRT was found to be 8 hr. Retention times shorter than this, caused washout of the organism and a drop in reactor performance (data not shown). It can be seen FIG. 2 that the ethanol concentration increases and the xylose concentration fall correspondingly during the start-up of the UIR reactors. After only 8 retention times the UIRs were stabilized at a HRT of 8 hr and an OLR of 31.4 g-COD/(L*d). This can be seen as an indication of build-up of Thermoanaerobacter HY10 on the granular sludge in the reactors.

Based on the preliminary chemostat results, further decrease in the HRT (below 8 hr) was expected to cause the washout of non-immobilized cells from the UIR reactors. However, the xylose conversion and the product concentrations remained unaffected by the gradual decreasing of the HRT to 4 hrs (FIG. 2). Thus, it is demonstrated that an active immobilized cell culture of T. HY10 can be established onto sterilised mesophilic granules of UIR reactors in less than 10 hydraulic retention times. It is also clear (FIG. 2) that the reactors were not under stress (organic overloading), as no immediate drop in performance was detected when the HRT was gradually decreased. A HRT of 4 hr corresponds to an OLR of 63.5 g-COD/(L*d), which is much higher than hydraulic retention times normally applied in UASB reactor operations of 30-45 g-COD/(L*d).

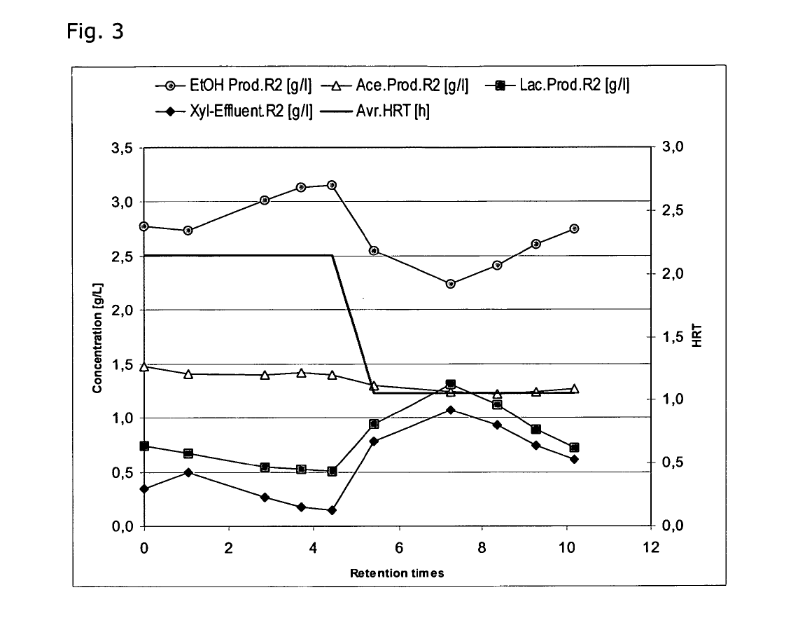

FIG. 3 shows the UIR reactor performance at extreme organic loading rates. Decreasing the HRT to 2 hr caused a drop in the performance of both reactors but after only 5 hydraulic retention times the UIRs recovered and were able to reach steady state converting 98.8% of the xylose at a yield of 0.32 g-ethanol/g-xylose. The drop in performance and the following recovery indicates that initially, there are not enough active Thermoanaerobacter HY10 immobilized on the granular sludge to cope with the increase of the OLR. However a build-up of active bacteria was completed in 15 hr to match the increased loading. After steady state is achieved at a HRT of 2 hr the loading of the reactors was further increased by reducing the HRT to only 1 hr corresponding to an OLR of 243 g-COD/(L*d) which is 6 times higher than stated in the literature as normal UASB loadings (Syutsubo et al., 1998). After an initial drop in performance the reactors recovered to convert the 93.8% of the xylose at a yield of 0.27 g-ethanol/g-xylose.

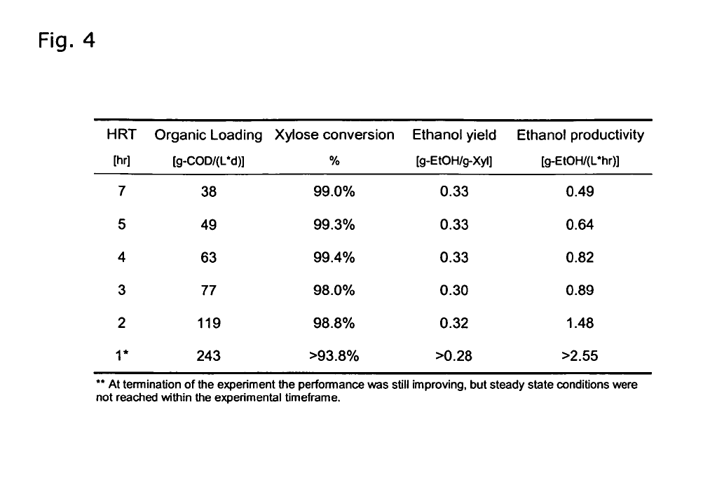

FIG. 4 shows the performance of the two UIR reactors operated under steady state at different OLRs. The presented figures are the average values obtained from the two identical operated reactors. Even though the two systems have been operated independently, the deviations of the presented data are less than 1% of the values. It is seen that at a HRT of 2 hr, the reactors were able to convert 98.8% of the xylose at a productivity of 1.48 g-ethanol/(L*hr) at an extreme loading rate of 119 g-COD/(L*d). At this loading, which is 3 times higher than normal applied in UASB reactors, the average productivity was 1.48 g-ethanol/(L*hr). The highest substrate conversion was 99.4% giving a yield of 0.33 g-ethanol/g-xylose and was achieved at HRT of 4 hr and OLR of 63 g-COD/(L*d). The composition of the produced gas was measured regularly during the experiments and was found to consist of 75-80% CO₂ and 20-25% H₂. The gas production ranged from 3 to 9 litres per day, depending on the organic loading.

The total experimental run time of the two reactor systems was 100 hydraulic retention times, which was achieved without any contamination. At the end of the experiment the integrity of the granules was unchanged, no suspended sludge or filamentous growth was observed. The shape of the granules had changed from black spheres to a little more flattened greyish shape.

### Example 2

### Immobilised BG1L1: Continuous Production of Ethanol by Fermentation of Xylose and Co-Fermentation of Xylose and Glucose Sugars

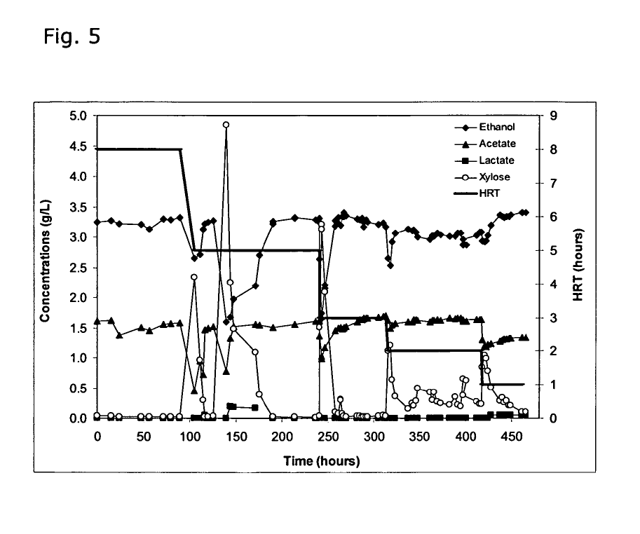

The potential of using an upflow immobilised reactor setup for continuous ethanol fermentation with the thermophilic anaerobic bacterium BG1L1, was investigated in a UIR as described above, operated at 70° C. (FIG. 5). The granules originated from the UASB reactor at Faxe waste water treatment plant (Denmark).

Before use, the reactor system was gassed for 15 minutes with N₂/CO₂ (4:1) to ensure anaerobic conditions and filled with BA medium with an initial xylose concentration of 10 g/l. The reactor was started-up in batch mode by inoculation with 80 ml of cell suspension with an optical density (OD₅₇₈) of 0.9-1. The batch mode of operation was kept during 24 hours to allow cells to attach and be immobilized onto on the carrier matrix. After the batch run, the system was switched to continuous mode applying a HRT of 8 hours and an up-flow velocity of 1 m/h.

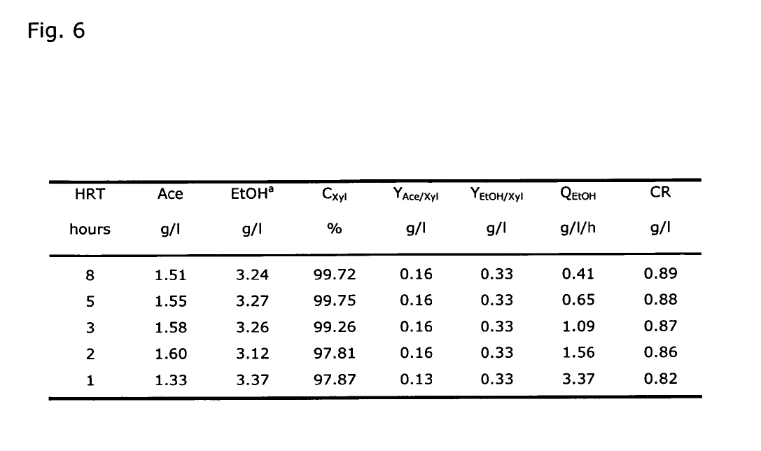

The effect of hydraulic retention time (HRT) on ethanol production and productivity was examined at a feed stream with 10 g/l xylose (FIG. 5). Product concentrations and xylose conversion were almost unaffected by gradually decreased HRT from 8 to 1 hour. Sugar conversion was higher than 97.8% yielding 0.33 g-ethanol/g-initial sugars and ethanol productivity gradually increased from 0.43 to 3.34 g/l/h (FIG. 6).

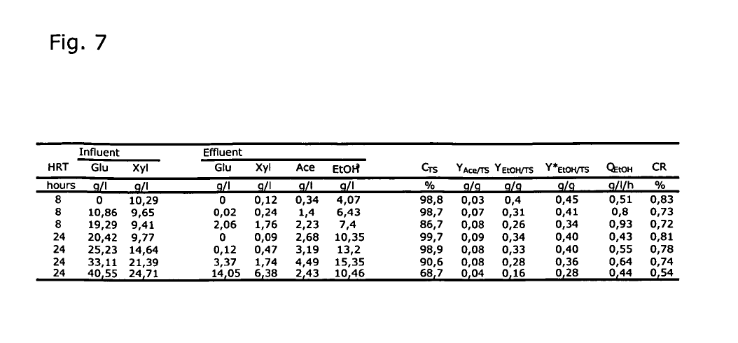

Economically, simultaneous co-fermentation of glucose and xylose could substantially reduce the cost of ethanol production from lignocellulose due to the potential high volumetric productivity because of shorter fermentation time. Hence, a second experiment was performed to investigate the co-fermentation of glucose and xylose (FIG. 7). Both sugars were simultaneously and effectively converted to ethanol with sugar utilization higher than 90.6% at sugar mixtures up to 54 g/l. At these sugar concentrations, the ethanol production increased gradually and the maximum ethanol concentration achieved was 15.35 g/l. Ethanol yields were 0.28-0.40 g-ethanol/g-initial sugars. The maximum ethanol productivity obtained was 1.1 g/l/h at HRT of 8 hours and 30 g/l sugars. The reactor was operated continuously for 140 days with no contamination and showed good long-term performance.

### Example 3

### Continuous Fermentation of Steam Exploded Wheat Straw Using Immobilised BG1L1

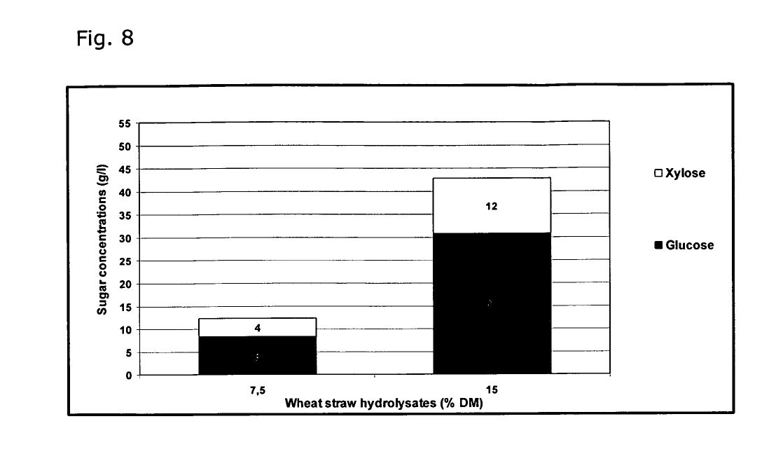

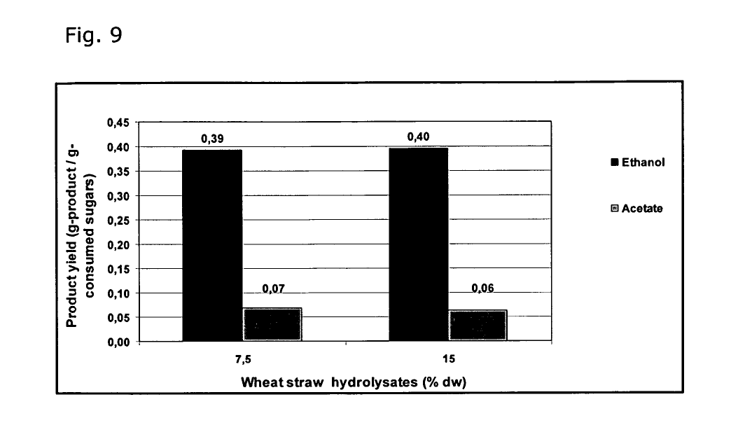

The potential of using an upflow immobilised reactor setup for continuous ethanol fermentation with the thermophilic anaerobic bacterium BG1L1, was investigated in a UIR as described above, operated at 70° C. (FIGS. 8 and 9) with steam exploded and enzyme treated wheat straw. The granules originated from the UASB reactor at Faxe waste water treatment plant (Denmark).

Steam exploded wheat straw hydrolysate (SEWS) was prepared by steam explosion followed by enzymatic hydrolysis (using Celluclast and Novozyme188 provided by Novozymes A/S) to release the constituent sugars, glucose and xylose. SEWS was provided by ELSAM, DK. The hydrolysate had a dry matter content of 23% (DM), and glucose and xylose were 57 g/l and 30 g/l, respectively. To counteract bacterial contamination, the SEWS hydrolysate medium was heated up to 121° C. for 1 min. Two SEWS suspensions were prepared by addition of respective volume of water given the desired concentrations of 7.5% and 15% DM corresponding to glucose-xylose mixtures of 12 and 43 g/l, respectively (FIG. 8). Even though the SEWS medium was un-detoxified, strain BG1L1 was capable of co-fermenting glucose and xylose efficiently with a relatively high ethanol yield of 0.39-0.4 g/g (FIG. 9). Glucose was completely utilized (>98%) for both tested SEWS suspensions, whereas xylose conversion decreased from 99% to 80% at 15% (DM) SEWS. Overall sugar conversion was higher than 90%. Acetate was the main by-product and remained relatively low during the entire fermentation (0.07-0.08 g/g) (FIG. 9).

During both experiments lasting for approximately 140 days, the reactor was checked regularly for contamination by purifying chromosomal DNA from reactor samples. No other species than BG1L1 was found. The deletion of the lactate dehydrogenase was also found to be stable as shown by sequencing of the lactate dehydrogenase region.

### Example 4

### Continuous Fermentation of Acid Pre-Treated Corn Stovers Using BG1L1

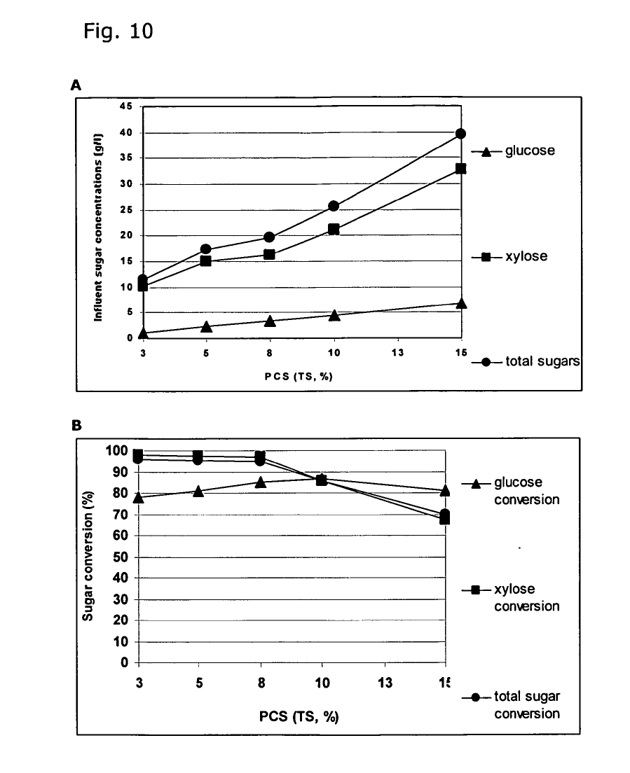

The potential of using an upflow immobilised reactor setup for continuous ethanol fermentation with the thermophilic anaerobic bacterium BG1L1 was investigated in a UIR as described above, operated at 70° C. (FIGS. 10, 11 and 12) with steam exploded and enzyme treated wheat straw. The granules originated from the UASB reactor at Faxe waste water treatment plant (Denmark).

Corn stover hydrolysate (PCS), prepared by dilute sulfuric acid hydrolysis, was provided by the National Renewable Energy Laboratory (Colden, Colo., USA). The hydrolysate had a total solids (TS) content of 30% (wt), and xylose, glucose and acetic acid concentrations were 67 g/l, 15 g/l and 14 g/l, respectively. Corn stover hydrolysate in concentrations of 2.5%-15% TS was used (FIG. 10A). Because of the low total sugar concentration in the hydrolysate suspensions of 2.5% and 5% TS, extra 5 g/l xylose was added to these suspensions to prevent eventual process problems caused by the relatively low sugar content.

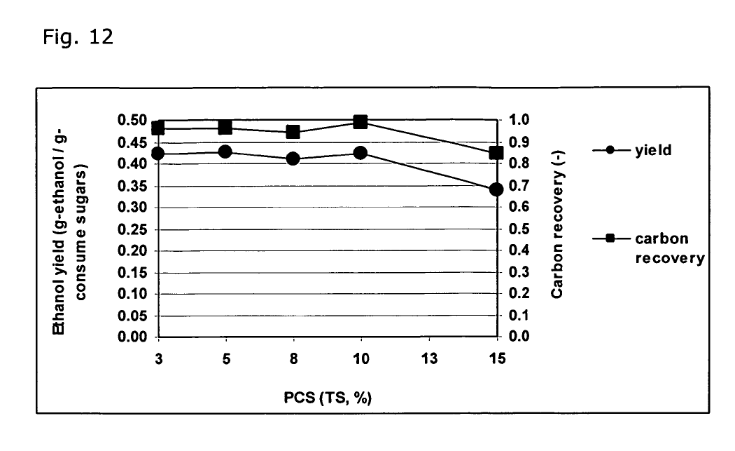

With PCS hydrolysate concentrations in the range of 2.5-10% TS, ethanol production increased gradually and relatively high and stable ethanol yields in a range of 0.41-0.43 g/g were obtained (FIG. 12). Almost complete sugar utilization (higher than 95%) was achieved for PCS of 2.5-7.5% TS, whereas at 10% (TS), the sugar conversion decreased to appr. 85% (FIG. 10B). At a PCS concentration of 15% TS, sugar conversion was 70% and relatively high ethanol yields of close to 0.35 g/g was obtained. The lower sugar conversion at 15% (TS) PCS (FIG. 10B) compared to other hydrolysate concentrations might be attributed to the growth and product inhibition caused by negative combination effect of high concentrations of acetate, other inhibitors present in the hydrolysate and salt accumulation resulted from based added for pH control (Lynd et al., 2001). However, the low ethanol yield at PCS of 15% TS (FIG. 12) was probably due to higher ethanol evaporation than expected, since the carbon recovery was low (CR<0.9).

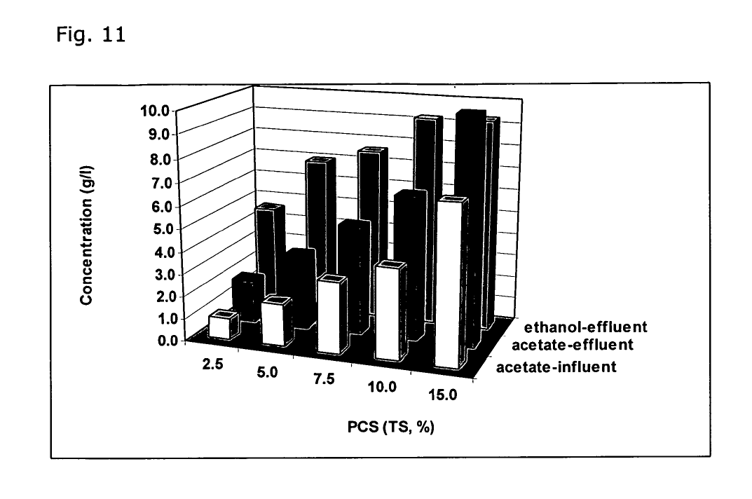

Acetate production increased from approximately 1 to 3.5 g/l (FIG. 11). However, because of high initial acetate concentrations (appr. 1-7 g/l) in the feed stream, a rather high concentration of nearly 10 g/l acetate was present in the effluent, which is significant with regard to the inhibitory effect of acetic acid to the fermentation. These results clearly show the high tolerance of the organism towards metabolic inhibitors present in undetoxified PCS in the shown reactor setup.

### Example 5

### Continuous Fermentation of Wet-Oxidized and Enzyme Treated Wheat Straw Using BG1L1

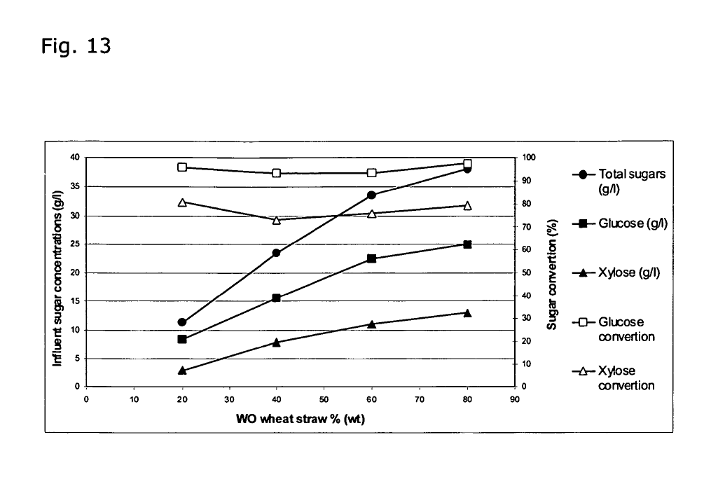

The potential of using a upflow immobilised reactor setup for continuous ethanol fermentation with the thermophilic anaerobic bacterium BG1L1, was investigated in a UIR as described above, operated at 70° C. (FIGS. 13, 14 and 15) with wet-oxidized and enzyme treated wheat straw. The granules originated from the UASB reactor at Faxe waste water treatment plant (Denmark).

As a feedstock, enzyme hydrolyzed WOWS (wet-oxidized wheat straw) material [200 g/l, 92.4% dry matter (DM)] in concentrations from 20 to 80% (wt) was used. Different WOWS suspensions (20-80% containing 3.7%-14.8% TS) were prepared by addition of water. Enzymatic hydrolysis was carried out with the commercial enzymes Celluclast, having an activity of 55.5 FPU/ml, and Novozyme 188, having an activity of 155 FBG/ml in a fixed relationship of 1.5 FBG/FPU. Enzyme mixture was loaded at FPU units per cellulose content corresponding to 28 FPU/g-cellulose. Enzymatic hydrolysis was carried out in a shaker at 50° C. for 4 days. After enzyme treatment the hydrolysate was centrifuged and the liquid fraction was further fermented into ethanol by Thermoanaerobacter BG1L1 at 70° C. (FIGS. 13, 14 and 15) and pH 7.0. The experiment was carried out with a gradual increase of WOWS hydrolysate concentrations from 20 to 80% (wt) (3.7%-14.8% TS), which corresponds to glucose-xylose mixture of 11-38 g/l sugars. Glucose and xylose conversion as well as sugar concentrations in the influent stream at various WOWS hydrolysate concentrations are given in FIG. 13.

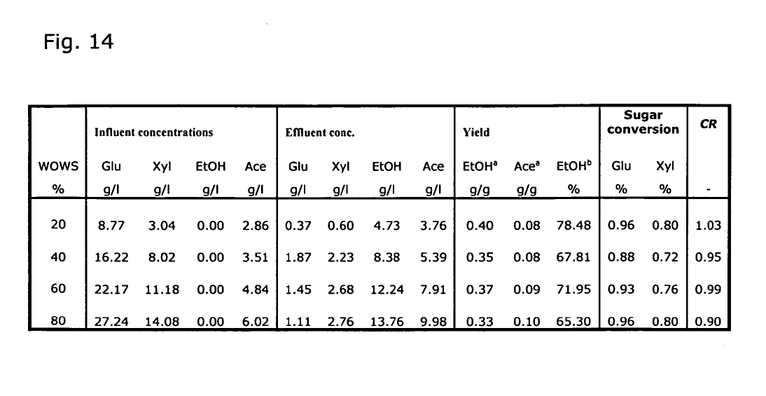

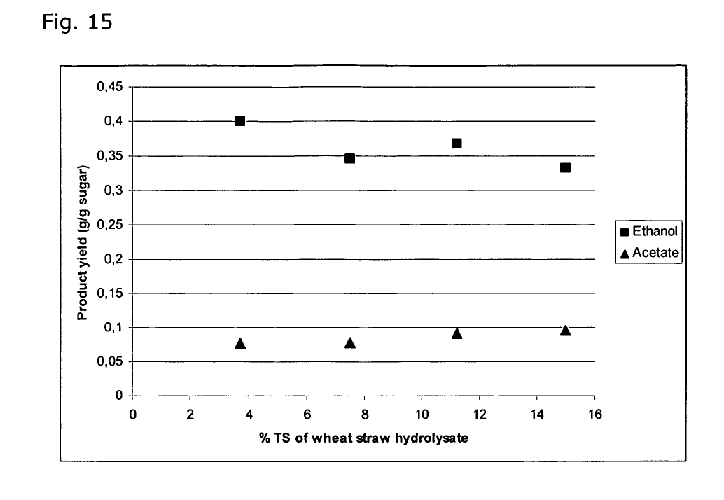

The sugar (glucose and xylose) conversion was not affected by the gradual increase in WOWS concentrations, which can be attributed to the low inhibitory effect of WOWS hydrolysate on the microbial activity in the immobilized reactor system. Glucose and xylose were simultaneously converted into ethanol. Nearly complete glucose utilization of 88-96% was obtained for all tested WOWS hydrolysate concentrations, whereas xylose conversion was in the range of 72-80%. In these experiments, ethanol yields in the range from 0.33-0.40 g/g were obtained (FIGS. 14 and 15). This corresponds to 65-78% of the theoretically possible yield.

### Example 6

### Effect of Exogenous Addition of Ethanol in a Fluidized Bed Reactor with Thermoanaerobacter BG1L1

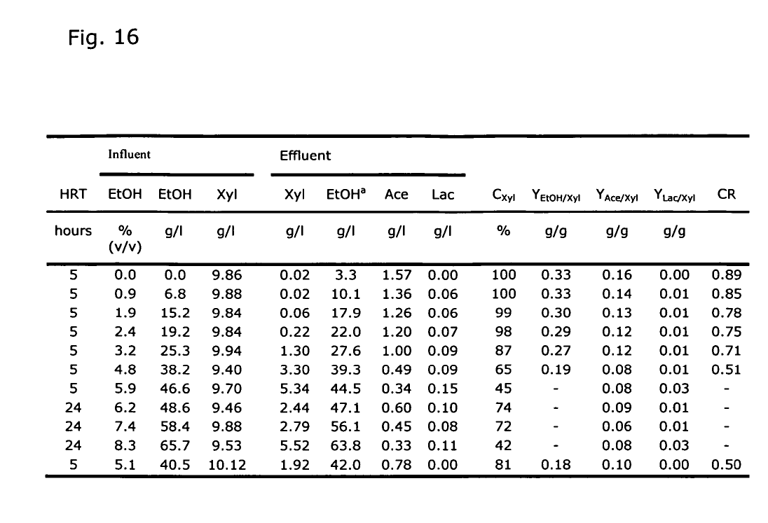

The ethanol tolerance of Thermoanaerobacter BG1L1 was examined at its optimum temperature for growth (70° C.) in a continuous immobilized fluidized bed reactor system (FIG. 16). The sludge granules for immobilisation originated from the UASB reactor at Faxe waste water treatment plant (Denmark).

The experiment was carried out with an influent containing 10 g/l xylose as a carbon source and ethanol up to 8.3%. The ethanol tolerance under steady-state conditions for each tested ethanol concentration was evaluated based on the fermentation activity of the strain, e.g. by xylose utilization and end-product formation of ethanol, acetate and lactate (FIG. 16). As can be seen, xylose is almost completely utilized (>98%) at initial ethanol concentrations up to 2.4% yielding ethanol in range of 0.29-0.33 g/g. Further increase in the added ethanol up to 5.9% had a clear effect on xylose utilization. Xylose conversion decreased linearly to 45%, which corresponds to an increase in effluent xylose concentration from 0.22 to 5.3 g/l. The ethanol yield (0.27 g/g), with an initial 3.2% ethanol, was comparable with those seen at lower added ethanol concentrations, whereas at 4.8% ethanol the yield dropped to 0.19 g/g. The low ethanol yield is probably due to loss of ethanol rather than inhibition of fermentation activity, because 49% of the carbon is missing (carbon recovery 0.51). The loss of carbon is probably caused by the combined effect of CO₂ stripping of ethanol and ethanol evaporation at the process temperature of 70° C., which is close to the boiling point of ethanol at 78° C. The loss of ethanol was more apparent when ethanol concentrations in the influent stream exceeded 4.8% as indicated by higher inlet than outlet ethanol concentrations for each steady state.

At 5.9% added ethanol and a HRT of 5 hours (FIG. 16), free cell biomass was visibly washed out (data not shown) and a transient increase in effluent xylose concentration from 3.3 to 6.9 g/l was seen (FIG. 16). Typically, a steady-state condition was established after 5 retention times, whereas at this ethanol concentration a new steady state was reached after a considerably longer period of time of approx. 30 retention times (˜6 days) (FIG. 16). At a new steady state, effluent xylose concentration (5.34 g/l) was lower than at the transient one and xylose utilization was 45%. The xylose conversion at a HRT of 5 hours was probably limited by slow microbial growth due to ethanol growth inhibition. In this regard, to achieve a higher xylose conversion, a longer-substrate residence time in the reactor is needed in order to enhance substrate availability. The fermentation continued further at a HRT of 24 hours. An increase in xylose conversion by 29% at ˜6% ethanol was observed. Further increases in added ethanol to 7.4% gave roughly the same xylose utilization of 72%. The highest ethanol concentration tested of 8.3% induced a dramatic drop in xylose conversion from 72% to 42%. The level of ethanol required to suppress microbial growth was not defined in this study. Thereafter, to test if improvement in strain performance could be seen derived from strain acclimation to higher ethanol concentrations, both the HRT and ethanol concentration were decreased respectively to 5 hours and 5% ethanol required for economically efficient product recovery. As can be seen from FIG. 16, xylose utilization improved by 16% compared to that at 4.8% ethanol at the beginning of experiment. An ethanol yield of 0.18 g/g was seen even though the amount of ethanol lost was not taken into consideration.

In summary, the use of the upflow immobilized reactor system makes it possible to ferment carbohydrates at a higher influent ethanol concentration than would be possible in batch fermentations. The absolute ethanol tolerance of BG1L1 in similar batch fermentations is approximately 4% v/v of exogenous ethanol, whereas continuous growth was observed even at an influent ethanol concentration of 8.3% in the continuous immobilized reactor system. The increased ethanol tolerance of the system is probably a combination of lower reactor concentrations of ethanol due to ethanol evaporation caused by gas stripping at the high temperatures and an effect of the cell immobilization leading to higher ethanol tolerance of the bacterium.

### REFERENCES

Andras, E., Kennedy, K. J., Richardson, D. A. 1989. Test for characterizing settleability of anaerobic sludge. Environ. Technol. Lett. 10 463-470
Bland R. J., Chen H C, Jewell W J, Bellamy W D, Zall R R: Continuous high rate production of ethanol by Zymomonas mobilis in an attached film expanded bed fermentor. Biotechnol Lett 1982, 4:323-328)
Bothast, R. J., Nichols, N. N., and Dien, B. S. (1999). Fermentations with new recombinant organisms. Biotechnol. Prog. 15, 867-875
De Bari, I., Cuna, D., Nanna, F., and Braccio, G. (2004). Ethanol production in immobilized-cell bioreactors from mixed sugar syrups and enzymatic hydrolysates of steam-exploded biomass. Applied Biochemistry and biotechnology 113-16, 539-557.
Gough, S., Brady, D., Nigam, P., Marchant, R., and Mchale, A. P. (1997). Production of ethanol from molasses at 45 degrees C. using alginate-immobilized Kluyveromyces marxianus imb3. Bioprocess Engineering 16, 389-392.
Kunduru M R, Pometto A L III: Continuous ethanol production by Zymomonas mobilis and Saccharomyces cerevisiae in biofilm reactors. J Ind Microbiol Biotechnol 1996, 16:249-256
Larsen, L., Nielsen, P., and Ahring, B. K. (1997). Thermoanaerobacter mathranii sp. nov., an ethanol-producing, extremely thermophilic anaerobic bacterium from a hot spring in Iceland. Arch. Microbiol. 168, 114-119.
Lynd, L. R., Ahn, H. J., Anderson, G., Hill, P., Kersey, D. S., and Klapatch, T. (1991). Thermophilic ethanol-production—investigation of ethanol yield and tolerance in continuous culture. Applied Biochemistry and biotechnology 28-9, 549-570.
Lynd, L. R., BASKARAN, S., and Casten, S. (2001). Salt accumulation resulting from base added for pH control, and not ethanol, limits growth of Thermoanaerobacterium thermosaccharolyticum HG-8 at elevated feed xylose concentrations in continuous culture. Biotechnology Progress 17, 118-125.
Schmidt, J. E. and Ahring, B. K. (1999). Immobilization patterns and dynamics of acetate-utilizing methanogens immobilized in sterile granular sludge in upflow anaerobic sludge blanket reactors. Applied And Environmental Microbiology 65, 1050-1054.

1. A process for the continuous production of a fermentation product by a fermentation process, wherein the fermentation product is produced by fermenting a carbohydrate solution in a biofilm reactor, said process comprising the steps of
(i) providing a solid support comprised of sterilised granular sludge,
(ii) admixing said solid support with an appropriate liquid medium comprising a carbohydrate solution to form a liquid growth medium,
(iii) contacting said liquid growth medium with an amount of microorganisms for a time period effective for cells of the microorganisms to attach to the surface of said solid support and form a film of microorganism cells on a substantial proportion of the surface of the support, and
(iv) cultivating said microorganisms under appropriate conditions to produce said fermentation product.

2. A process according to claim 1, wherein said sterilised granular sludge is derived from a bioreactor selected from the group consisting of a fluidized bed reactor (FBR), a gaslift reactor, an upflow anaerobic sludge blanket reactor (UASBR), an upflow staged sludge bed (USSB) reactor, expanded granular sludge bed (EGSB) reactor, internal circulation reactor, upflow anaerobic filter process (UAFP), and an anaerobic fluidized-bed reactor (AFBR).

3. A process according to claim 1, wherein the sterilised granular sludge is comprised of spherical granules with a diameter in the range of about 0.10 to 5 mm.

4. A process according to claim 1, wherein the sterilised granular sludge is comprised of spherical granules with a diameter in the range of about 2 to 4 mm.

5. A process according to claim 1, wherein the sterilised granular sludge is comprised of spherical granules having a settling velocity in the range of about 18 to 100 m/h.

6. A process according to claim 5, wherein the settling velocity is up to about 20 m/h.

7. A process according to claim 5, wherein the settling velocity is from about 20 to 50 m/h.

8. A process according to claim 5, wherein the settling velocity is at least about 50 m/h.

9. A process according to claim 1, wherein the sterilised granular sludge comprises volatile suspended solids in the range of 5-15% weight.

10. A process according to claim 1, wherein the biofilm reactor is selected from the group consisting of fluidized bed reactors (FBR), gaslift reactor and upflow anaerobic sludge blanket reactors (UASBR), upflow staged sludge bed (USSB) reactors, expanded granular sludge bed (EGSB) reactors, internal circulation reactors, and upflow anaerobic filter process (UAFP).

11. A process according to claim 1, wherein the carbohydrate solution comprises carbohydrates selected from the group consisting of monosaccharides, oligosaccharides and polysaccharides.

12. A process according to claim 11, wherein the polysaccharides are selected from the group consisting of starch, lignocellulose, cellulose, hemicellulose, chitin, pectin, glycogen, xylan, glucuronoxylan, arabinoxylan, arabinogalactan, glucomannan, xyloglucan, and galactomannan.

13. A process according to claim 12, wherein the lignocellulose is derived from a lignocellulosic biomass material.

14. A process according to claim 13, wherein the lignocellulosic biomass material is present in the liquid growth medium at a dry-matter content of at least 10% wt/wt.

15. A process according to claim 13, wherein the lignocellulosic biomass material has been subjected to a pre-treatment step selected from acid hydrolysis, steam explosion, wet oxidation and enzymatic hydrolysis.

16. A process according to claim 1, wherein the fermentation product is selected from the group consisting of an acid, an alcohol, a ketone and hydrogen.

17. A process according to claim 16, wherein the alcohol is selected from the group consisting of ethanol, butanol, propanol, methanol, propanediol and butanediol.

18. A process according to claim 16, wherein the acid is selected from the group consisting of lactic acid, proprionate, acetate, succinate, butyrate and formate.

19. A process according to claim 16, wherein the ketone is acetone.

20. A process according to claim 1, wherein the microorganism belongs to a genus selected from Saccharomyces, Thermoanaerobacter, Clostridium, Moorella, Lactobacillus, Aspergillus, Pichia, Zymomonas, Zymobacter, Pseudomonas, Escherichia, Acetobacterium, Propionibacterium og Acetogenium.

21. A process according to claim 20, wherein the Thermoanaerobacter is selected from the group consisting of Thermoanaerobacter acetoethylicus, Thermoanaerobacter brockii, Thermoanaerobacter brockii subsp. brockii, Thermoanaerobacter brockii subsp. finnii, Thermoanaerobacter brockii subsp. lactiethylicus, Thermoanaerobacter ethanolicus, Thermoanaerobacter finnii, Thermoanaerobacter italicus, Thermoanaerobacter kivui, Thermoanaerobacter lacticus, Thermoanaerobacter mathranii, Thermoanaerobacter pacificus, Thermoanaerobacter siderophilus, Thermoanaerobacter subterraneus, Thermoanaerobacter sulfurophilus, Thermoanaerobacter tengcongensis, Thermoanaerobacter thermocopriae, Thermoanaerobacter thermohydrosulfuricus, Thermoanaerobacter wiegelii, Thermoanaerobacter yonseiensis.

22. A process according to claim 21, wherein the Thermoanaerobacter mathranii strain is selected from HY10 (DSMZ accession number 14578), BG1 (DSMZ Accession number 18280) and mutants thereof.

23. A process according to claim 22, wherein the mutant is selected from BG1L1 (DSMZ Accession number 18283), BG1PF1 (DSMZ Accession number 18282) and BG1H1 (DSMZ Accession number 18281).

24. A process according to claim 1, which is performed under strict anaerobic conditions.

25. A process according to claim 1, which is operated at a temperature in the range of about 40-95° C.

26. A process according to claim 1, which is performed in an upflow anaerobic sludge blanket reactor operated at a hydraulic retention time (HRT) in the range of about 5 to 72 hours.

27. A process according to claim 1, further comprising the step of retrieving the fermentation product.

28. A process according to claim 1, wherein the sterilised granular sludge comprises volatile suspended solids in the range of 7-12% weight.

29. A process according to claim 1, wherein the sterilised granular sludge comprises volatile suspended solids in the range of 8-11% weight.

30. A process according to claim 13, wherein the lignocellulosic biomass material is selected from the group consisting of straw, hay, garden refuse, house-hold waste, wood, fruit hulls, seed hulls, corn hulls, oat hulls, soy hulls, corn fibres, stovers, milkweed pods, leaves, seeds, fruit, grass, wood, paper, algae, cotton, hemp, flax, jute, ramie, kapok, bagasse, mash, distillers grains, oil palm, corn, sugar cane and sugar beet.

31. A process according to claim 13, wherein the lignocellulosic biomass material is present in the liquid growth medium at a dry-matter content of at least 15% wt/wt.

32. A process according to claim 13, wherein the lignocellulosic biomass material is present in the liquid growth medium at a dry-matter content of at least 20% wt/wt.

33. A process according to claim 13, wherein the lignocellulosic biomass material is present in the liquid growth medium at a dry-matter content of at least 25% wt/wt.

34. A process according to claim 13, wherein the lignocellulosic biomass material is present in the liquid growth medium at a dry-matter content of at least 35% wt/wt.

35. A process according to claim 1, which is operated at a temperature in the range of about 50-90° C.

36. A process according to claim 1, which is operated at a temperature in the range of about 60-85° C.

37. A process according to claim 1, which is operated at a temperature in the range of about 65-75° C.

38. A process according to claim 1, which is performed in an upflow anaerobic sludge blanket reactor operated at a hydraulic retention time (HRT) in the range of about 7 to 12 hours.

39. A process according to claim 1, which is performed in an upflow anaerobic sludge blanket reactor operated at a hydraulic retention time (HRT) in the range of about 8 to 10 hours.
